(19) United States
(12) Patent Application Publication
(10) Pub. No.: US 2006/0057101 A1
(43) Pub. Date: Mar. 16, 2006

(54) German cockroach attractant

(76) Inventors: Wendell Roelofs, Geneva, NY (US); Coby Schal, Cary, NC (US); Francis X. Webster, Syracuse, NY (US); Satoshi Nojima, Raleigh, NC (US)

Correspondence Address: Michael L. Goldman, Esq.;Nixon Peabody LLP, Clinton Square,, P.O. Box 31051, Rochester, NY, 14603-1051, US

(21) Appl. No.: 11/178,833

(22) Filed: Jul. 11, 2005

Related U.S. Application Data
(60) Provisional application No. 60/588,049, filed on Jul. 14, 2004.

(51) Int. Cl. A01N 25/00; C07C 50/38

(52) U.S. Cl. 424/84; 552/309

(57) ABSTRACT

A compound of the formula I 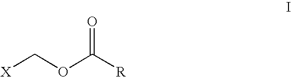
where R represents unsubstituted or substituted, alkyl, cycloalkyl, alkenyl, alkynyl, or aryl, and X represents 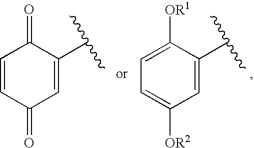
where R¹ and R² are each H or alkyl, is disclosed. Compounds of formula I are particularly useful as an attractant for cockroaches, namely, Blattella germanica. Synthesis of the compounds, methods of controlling cockroach populations, and of disrupting cockroach mating are also disclosed.

This application claims the priority benefit of U.S. Provisional Patent Application Ser. No. 60/588,049, filed Jul. 14, 2004, which is hereby incorporated by reference in its entirety.

### FIELD OF THE INVENTION

The present invention relates to attractants for cockroaches, and more particularly, attractants for the German cockroach, Blattella germanica.

### BACKGROUND OF THE INVENTION

Cockroaches have long been recognized as pests of economic, medical, veterinary, and aesthetic concern. Brenner, “Medical and Economic Significance,” in Rust, ed., Understanding and Controlling the German Cockroach, New York: Oxford University Press, pp. 77-92 (1995). The German cockroach, Blattella germanica, is the most prominent and most important indoor structural pest worldwide. Schal et al., “Integrated Suppression of Synanthropic Cockroaches,” Annual Review of Entomology 35:521-551 (1990); Brenner, “Medical and Economic Significance,” in Rust, ed., Understanding and Controlling the German Cockroach, New York: Oxford University Press, pp. 77-92 (1995). It has been shown to carry a number of pathogenic microorganisms (Zurek et al., “Evaluation of the German Cockroach, Blattella Germanica, as a Vector of Verotoxigenic Escherichia Coli F 18 in Confined Swine Production,” Veterinary Microbiology 101:263-267 (2004)), and cockroaches are recognized as a significant etiological agent in allergic respiratory diseases, especially bronchial asthma.

A number of NIH reports indicate that the prevalence and incidence of asthma have been rising at alarming rates. Platts-Mills et al., “Indoor Versus Outdoor Allergens in Allergic Respiratory Disease,” Curr Opin Immunol 10:634-639 (1998). Of the approximately 400,000 yearly emergency room admissions of adults with asthma, approximately 200,000 are associated with mite, cat, or cockroach allergens. Gelber et al., “Sensitization and Exposure to Indoor Allergens as Risk Factors for Asthma Among Patients Presenting to Hospital,” Am. Rev. Respir. Dis. 147:573-578 (1993). In the National Cooperative Inner-City Asthma Study (“NCICAS”), 37% of the children studied were allergic to cockroaches because of chronic exposure to cockroaches. Rosenstreich et al., “The Role of Cockroach Allergy and Exposure to Cockroach Allergen in Causing Morbidity Among Inner-City Children With Asthma,” N. Engl. J. Med. 336:1356-1363 (1997). According to the study, these children had a 3.4-fold higher rate of hospitalization than other study cohorts, made 78% more asthma-related unscheduled visits to health care providers, awoke more nights, and missed more school days. They also had more days of wheezing, and their caregivers were forced to change their plans more often (e.g., missed work). Other studies have confirmed that cockroach allergen level is a good predictor of repeated wheeze and asthma. These measures of morbidity due to asthma highlight the medical and economic costs of cockroach infestations to society. However, cockroach-induced allergies and asthma are not strictly an inner-city phenomenon. There is a clear link between housing quality, cockroach infestation, and allergic disease, in both the inner city and the suburbs (Couture et al., “Exposure To Indoor Allergens of 7th and 8th Grade Children in Central Virginia—Relationship to Sensitization of African-American and Caucasian Children,” Journal of Allergy and Clinical Immunology 95:215-215 (1995)), and indeed, across various societies. Rona R., “Asthma and Poverty,” Thorax 55:239-244 (2000).

These studies suggest that the best approach to reducing bronchial hyperreactivity is prolonged avoidance of allergens. Indeed, cockroach control, coupled with extensive cleaning, results in significant reductions in cockroach allergens in settled dust. Sarpong et al., “Socioeconomic Status and Race as Risk Factors for Cockroach Allergen Exposure and Sensitization in Children With Asthma,” J. Allergy Clin. Immunol. 97:1393-1401 (1996a); Gergen et al., “Results of the National Cooperative Inner-City Asthma Study (NCICAS) Environmental Intervention to Reduce Cockroach Allergen Exposure in Inner-City Homes,” J. Allergy Clin. Immunol. 103:501-506 (1999); Williams et al., “Cockroach Extermination Does Not Rapidly Reduce Allergen in Settled Dust,” J Allergy Clin Immunol 104:702-3 (1999); Eggleston et al., “Removal of Cockroach Allergen From Inner-City Homes,” J Allergy Clin Immunol 104:842-6 (1999); Arbes et al., “Abatement of Cockroach Allergen (Bla g 1) in Low-Income, Urban Housing—a Randomized Controlled Trial,” Journal of Allergy and Clinical Immunology 112: 339-345 (2003). However, this effect is rarely sustained over time, mainly because cockroaches re-infest the homes. Recent studies have, in fact, shown that reduction of cockroach infestations can significantly reduce environmental allergens. Arbes et al., “Abatement of Cockroach Allergens (Bla g 1 and Bla g 2) in Low-Income, Urban Housing: Month 12 Continuation Results,” Journal of Allergy and Clinical Immunology 113:109-114 (2004).

Because of their movement between waste (e.g., sewers, manure) and food materials, German cockroaches can acquire, carry, and transfer pathogens either mechanically or in their digestive system. Cockroaches have a significant vector potential because a high percentage of cockroaches in large infestations may be able to transmit such infectants as Salmonella, other microbes, helminths, fungi, protozoans, and viruses. Research at North Carolina State University demonstrated that cockroaches could acquire S. typhimurium from an infected food source, and infect non-contaminated cockroaches, food, and water through contact. Kopanic et al., “Cockroaches As Vectors of Salmonella: Laboratory and Field Trials,” J. Food Protection 57:125-132 (1994). Most alarming were the study's findings that cockroaches collected in a commercial poultry feed mill and hatchery were contaminated with Salmonella, and that they efficiently transferred bacteria to poultry eggs.

In another study, vector competence of German cockroaches for one of the most important porcine bacterial pathogens, verotoxigenic Escherichia coli F18, was evaluated using a culturing approach followed by multiplex PCR. In addition, the populations of fecal coliforms from the feces of piglets and cockroaches collected from a swine nursery were assessed. Viable and virulent cells of E. coli F 18 were detected in cockroach feces for up to 8 days after the initial exposure. The population of fecal coliforms in cockroach feces was high and comparable to that of the piglet feces. This study demonstrates that cockroaches may serve as important mechanical vectors of pathogenic E. coli. Accordingly, integrated management of cockroach populations should be incorporated into the disease prevention and control programs in the swine industry.

Effective suppression of cockroach populations is therefore needed to alleviate health-related problems, and is, in fact, mandated by federal and state regulations. Cockroaches are generally controlled with scheduled broadcast applications of broad-spectrum synthetic insecticides or with baits. Wickham, “Conventional Insecticides,” in Rust, ed., Understanding and Controlling the German Cockroach, Oxford Univ. Press., pp. 109-148 (1995); Koehler, “Chemical Systems Approach to Pest Control,” in Rust, ed., Understanding and Controlling the German Cockroach, Oxford Univ. Press, pp. 287-324 (1995). However, synthetic pesticides, while reducing pest populations if properly applied, can expose the applicator and the consumer to health and environmental risks. Alternatives to pesticides are therefore needed and it is imperative that safe, effective, and environmentally compatible insect control techniques be developed and incorporated into sustainable integrated pest management programs (“IPM”). Attractants, such as foods or pheremones, are one such alternative.

There are several major potential uses of effective attractants in cockroach control. Attractants may be used, for example, in the detection and monitoring of cockroach populations, for mass trapping, with insecticides, in biological control programs, and the like. Detection and monitoring are both fundamental components of the decision-making component of IPM. Owens, “Detection and Monitoring,” Rust, ed., Understanding and Controlling the German Cockroach, Oxford University Press, pp. 93-108 (1995). Because pest control professionals are relatively distant from the “account” they service and are much less intimately associated with the pest population than are farmers, it is more difficult for them to evaluate the pest problem. Detection and monitoring with traps offers a powerful tool in a well-planned inspection. Also, because the cockroach population is distributed unevenly in a highly heterogeneous environment (unlike agricultural monocultures), the inspection for cockroaches relies on intensive trapping. The use of numerous baited traps has the potential to locate infestations, but current traps are relatively inefficient in low cockroach densities because of their limited capture space, and because they do not sample cockroaches that retreat to deep, insecticide-free shelters. Nalyanya et al., “Attractiveness of Insecticide Baits for Cockroach Control (Dictyoptera: Blattellidae): Laboratory and Field Studies,” Journal of Economic Entomology 94:686-693 (2001). Moreover, in many situations, such as in hospitals and laboratories, the threshold for implementing control against cockroaches may be a single sighting (for example, on an operating table), thus requiring highly sensitive detection tools.

With regard to mass trapping of cockroaches, several studies have documented that mass removal of cockroaches can effectively reduce infestations. Mass removal is currently done with specialized vacuum cleaners. Traps baited with effective attractants may serve to remove unwanted cockroaches from structures. Effective attractants can be incorporated into insecticide formulations to both reduce the inherent repellency of such formulations and to bring cockroaches closer to the pesticide.

Attractants may be used to disseminate biological control agents against cockroaches. Various entomopathogenic fungi and nematodes must be contained in baits to maintain their virulence. Pheromones may be used to attract and inoculate cockroaches, which in turn transmit the pathogen to other cockroaches. Nalyanya et al., “Integration of Repellents, Attractants, and Insecticides in a “Push-Pull” Strategy for Managing German Cockroach (Dictyoptera: Blattellidae) Populations,” Journal of Medical Entomology 37:427-434 (2000) showed that repellents could be integrated with attractive baits and insecticides to manipulate the distribution of cockroaches in a “push-pull” system. Ultimately, this results in more effective, faster removal of cockroaches from structures. The lack of efficient attractants and trapping methods for cockroaches is probably the most significant single factor contributing to a heavy reliance on scheduled applications of insecticides.

In recent years, pheromones have been recognized as useful components of a successful pest control program. A pheromone is generally defined as a chemical substance secreted by living organisms, including insects, to convey information or to produce a specific response in other individuals of the same species. Sex pheromones typically take the form of a complex, volatile blend of compounds, typically excreted during the mating cycle. As such, sex pheromones often serve as “attractants”; that is, the pheromone attracts members of the same species to the site of the pheromone emission. Sex pheromones are excellent, clean monitoring tools for the pest control operator and the food sanitarian in restaurants, food processing facilities, warehouses, and similar facilities and for the farmer in confined animal production systems. This approach is particularly important in areas such as hospitals, schools, zoos, and livestock barns, where conventional insecticide usage is restricted. Availability of an effective pheromone promises to facilitate the development of environmentally compatible pest management programs for structural ecosystems.

There are several pheromones used by cockroaches, including those used at a distance (volatile), at close range (mostly male tergal secretions), and by contact only (cuticular components). Long distance pheromones are practically useful in pest control programs. Chow et al. (“Attraction Responses of the American Cockroach to Synthetic Periplanone-B,” J. Chem. Ecoli. 7:265-272 (1981)) describe the use of periplanone B as an attractant for American cockroaches. U.S. Pat. No. 5,296,220 to Roelofs et al. describes a group of pyran compounds that are effective attractants particularly useful for male cockroaches of the brown-banded type. Liang et al. (“Field and Laboratory Evaluation of Female Sex Pheromone for Detection, Monitoring, and Management of Brownbanded Cockroaches (Dictyoptera: Blattellidae),” J. Econ. Entomol. 91: 480-485 (1998)) showed that supellapyrone, the synthetic sex pheromone of the female brownbanded cockroach, was highly attractive to males in the field. Jar traps with pheromone caught more total cockroaches than two commercial baits that presumably contain attractants. Combination of the food attractant and pheromone resulted in further increases in trap catch. The pheromone increased the number of adult males in all treatments by 6-28 times relative to the respective controls.

Currently, the majority of traps against German cockroaches utilize food attractants, or nothing at all. While there have been reports of aggregation pheromones in German cockroaches, their chemical identification remains uncertain. Nalyanya et al., “Evaluation of Attractants for Monitoring Populations of the German Cockroach (Dictyoptera: Blattellidae),” Journal of Economic Entomology 94:208-214 (2001). Currently, there are no effective sex pheromones available for German cockroach control. As recent as 1993, the prevailing view was that mate-finding in B. germanica involves random encounters between females and males, facilitated by the clumped distribution of the insects, which is mediated by aggregation pheromones. Schal et al., “Behavioural Ecology of Cockroaches,” Biological Reviews of the Cambridge Philosophical Society 59:209-254 (1984). Upon contact, courtship in the German cockroach is mediated by a female contact sex pheromone (Roth et al., “A Study of Cockroach Behavior,” Amer. Midland Naturalist 47:66-129 (1952)) that is composed of at least four components that elicit male courtship wing-raising responses (Nishida et al., “Female Sex Pheromone of the German Cockroach, Blattella Germanica,” Mem. Coll. Agric. 122:1-24 (1983); Schal et al., “A New Component of the Sex Pheromone of Blattella Germanica (Dictyoptera: Blattellidae), and Interaction with other Pheromone Components,” Journal of Chemical Ecology 16:1997-2008 (1990)). In 1993, however, it was discovered that sexually receptive, virgin German cockroach females exhibit a characteristic calling behavior during which a volatile sex pheromone is released. Liang et al., “Volatile Sex Pheromone in the Female German Cockroach,” Experientia 49:324-328 (1993). It was suggested that calling and the emission of a volatile sex pheromone serves to attract males from a distance, as well as to potentiate responses to contact sex pheromone in aggregations. Using behavioral and electrophysiological assays, the pheromone-producing gland was localized to the last abdominal tergite, and extensive endocrine studies were conducted on the regulation of pheromone production. Liang et al., “Ultrastructure and Maturation of a Sex Pheromone Gland in the Female German Cockroach, Blattella Germanica,” Tissue & Cell 25:763-776 (1993).

The present invention is directed to overcoming these and other deficiencies in the art.

### SUMMARY OF THE INVENTION

One aspect of the present invention is a compound of formula I 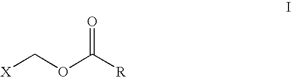
where R represents unsubstituted or substituted, alkyl, cycloalkyl, alkenyl, alkynyl, or aryl, and X represents 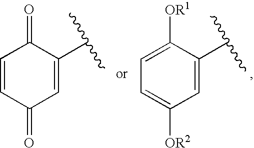
where R¹and R² are each H or alkyl.

Another aspect of the present invention is a method of producing a compound of formula I. The method involves reacting a compound of formula II 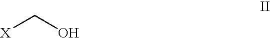
where X represents 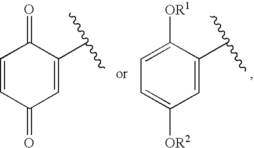
where R¹ and R² are each H or alkyl, under conditions effective to produce a compound of formula I.

A further aspect of the present invention is an unsubstituted or substituted, alkyl, cycloalkyl, alkenyl, alkynyl, or aromatic ester of unsubstituted or substituted, gentisyl alcohol or gentisyl quinone.

Yet another aspect of the present invention is a method of attracting cockroaches, which involves exposing cockroaches to a trap charged with an effective attractant amount of a compound of formula I.

Still another aspect of the present invention is a method of disrupting cockroach mating, which involves exposing a cockroach population to a compound of formula I in an amount sufficient to disrupt mating.

Yet a further aspect of the present invention is a cockroach trap that includes a support, a means for restraining cockroaches, and a cockroach attractant amount of a compound of formula I.

Still a further aspect of the present invention is a cockroach control system that contains an insecticide and a cockroach attractant of formula I.

Still a further aspect of the present invention is a cockroach control system that contains a biocontrol agent and a cockroach attractant of formula I.

The compounds of the present invention are based upon a newly discovered sex pheromone derived from Blattella germanica, and have the potential to revolutionize the way cockroach control is done. Currently deployed traps are so ineffective that pest control professionals routinely use insecticides in conjunction with the traps, thus showing their lack of trust in the efficacy of their traps. In addition, because adult males are much more mobile than any other stage of the German cockroach, the compounds of the present invention also provide highly sensitive detection systems.

### BRIEF DESCRIPTION OF THE DRAWINGS

FIGS. 1 and 2 show comparative data for the natural pheromone product and the synthetic compound, respectively.

FIG. 3 is a graphic representation of behavioral responses of male German cockroaches to one female equivalent of a crude extract of many virgin females and to various doses of the synthetic pheromone, gentisyl quinone isovalerate. The pheromone was applied in 20 μl of methylene chloride to a filter paper disc and presented to single males acclimated in the two-choice olfactometer vs. a methylene chloride control filter paper. Behavioral response is defined as quiescent males that were activated and ran out of their cage. Each assay consisted of testing 10-16 males in individual olfactometers and the assays were replicated 3-18 times. The mean responses and SEM are indicated.

FIG. 4 is a graphic representation of behavioral responses of male German cockroaches to one female equivalent of a crude extract of many virgin females and to various doses of the synthetic pheromone, gentisyl quinone isovalerate. Procedures are as in FIG. 3, but reported is the latency in seconds before males left their cages.

FIG. 5 is a graphic representation of trap catches of German cockroaches using various doses of the synthetic pheromone, gentisyl quinone isovalerate. The pheromone was applied in 50 μl of methylene chloride to a rubber septum, allowed to dry, and then another 50 μl of methylene chloride was applied to the rubber septum. The septum was hung inside a 1 pint wide-mouth mason jar whose inside wall was treated with a thin layer of petroleum jelly (to prevent trapped cockroaches from escaping). Each mason jar was wrapped in a cloth sleeve to facilitate cockroaches climbing. Traps were placed in a completely randomized Latin Square design along walls of farrowing rooms in a cockroach infested swine farm. Treatments (7 doses) were rotated daily so that all positions along the wall received all treatments over 7 consecutive nights. Each mean represents 35 replicates. Data analysis is by multi-way ANOVA on log transformed data, followed by LSD mean separation.

FIG. 6 is a graphic extension of the dose-response experiment shown in FIG. 5. All procedures are as in FIG. 5, except only 3 treatments (3 doses) were rotated daily so that all positions along the wall received all treatments over 3 consecutive nights. Each mean represents 19 replicates. Data analysis is as in FIG. 5.

### DETAILED DESCRIPTION OF THE INVENTION

The present invention relates to compounds useful as attractants for cockroaches. In one aspect, the compound is a compound of formula I 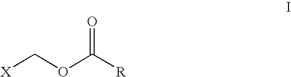
where R represents unsubstituted or substituted, alkyl, cycloalkyl, alkenyl, alkynyl, or aryl, and X represents 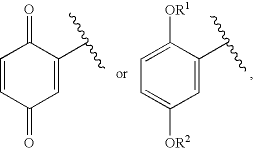
where R¹ and R² are each H or alkyl.

In accordance with the present invention, alkyl means a linear or branched saturated hydrocarbon group having 1 to 20 carbon atoms. Lower alkyl means an alkyl group having 1 to 10, preferably 1 to 8, and more preferably, 1 to 6 carbon atoms. Alkyl includes, for example, methyl, ethyl, n-propyl, isopropyl, n-butyl, isobutyl, sec-butyl, tert-butyl pentyl, iso-amyl hexyl, octyl groups and the like. Substituted alkyl means an alkyl group where at least one hydrogen atom attached to an aliphatic carbon is replaced with a substituent such as, for example, alkyl, amino, alkoxy, hydroxy, aryl, heteroaryl, aryloxy, cyano, carboxy, alkoxycarbonyl, monoakylamino, alkyloxy, cyanoalkyl, cycloalkyl, alkylthio, alkylsulfinyl, alkylsulfonyl, arylthio, carboxyalkyl, alkoxycarbonylalkyl, haloalkyl, acylamino, dialkylamino, cyclicamino, halogen, or nitro. Such substituent groups can include, for example, methyl, isopropyl, methoxy, ethoxy, propoxy amino, methylamino, phenyl, naphthyl groups, chlorine, fluorine and the like.

Cycloalkyl means a saturated hydrocarbon ring group having 3 to 12, preferably 3 to 8 carbon atoms. Lower cycloalkyl means a cycloalkyl group having 3 to 6 carbon atoms. Cycloalkyl includes, for example, cyclopropyl, cyclobutyl, cyclopentyl, cyclohexyl, norbornyl, adamantyl, myrtanyl groups and the like. Substituted cycloalkyl means a cycloalkyl group where at least one hydrogen atom attached to a ring carbon atom is replaced with a substituent group as previously described herein.

Alkenyl means a linear or branched, unsaturated acyclic hydrocarbon group having 2 to 20, preferably 2 to 12 carbon atoms, and containing at least one double bond. Lower alkenyl means an alkenyl group having 2 to 10, preferably 2 to 8, and more preferably 2 to 6 carbon atoms. Alkenyl includes, for example, propenyl, isopropenyl, 1-butenyl, 2-butenyl, 1-isobutenyl, 1,3-butadienyl, 2-methylbuten-1-yl, 3-methylbuten-1-yl, 1-pentenyl, 1-hexenyl, 1-heptenyl, 1-octenyl, vinyl, and the like. Substituted alkenyl means an alkenyl group where at least one hydrogen atom attached to an aliphatic carbon atom is replaced with a substituent group as previously described herein.

Alkynyl means a linear or branched, unsaturated acyclic hydrocarbon group having 2 to 20, preferably 2 to 12, more preferably 2 to 8 carbon atoms, and containing at least one triple bond. Alkynyl includes, for example, ethynyl, propynyl, 1-butynyl, 2-butynyl, 1-pentynyl, 1-heptynyl, 1-octynyl, and the like. Substituted alkynyl means an alkynyl group where at least one hydrogen atom attached to an aliphatic carbon atom is replaced with a substituent group as previously described herein.

Aryl means an aromatic monocyclic or fused hydrocarbon ring group having 4 to 18, preferably 4 to 16, more preferably 6 to 12, and most preferably 6 to 10 carbon atoms. Aryl includes, for example, phenyl, biphenyl, 3,4-dichlorophenyl, naphthyl, and the like. Substituted aryl means an aryl group where at least one methine hydrogen atom attached to an aromatic carbon is replaced with a substituent group as previously described herein.

Preferably, R is alkyl, more preferably, isobutyl. Preferably, X represents

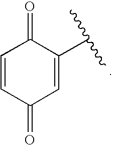

Compounds of formula I are, for example, unsubstituted or substituted, alkyl, cycloalkyl, alkenyl, alkynyl, or aromatic esters of gentisyl alcohol or gentisyl quinone.

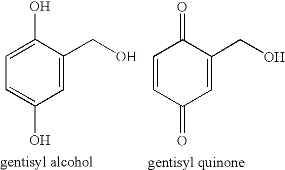

Accordingly, compounds of the present invention include, for example:

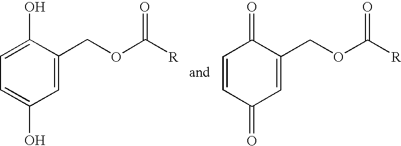

A particularly useful compound of formula I is gentisyl quinone isovalerate:

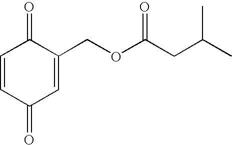

Compounds of the present invention can also include compounds of formula I wherein various substitutions are made on the basic ring structure. Accordingly, compounds of the present invention include, for example, unsubstituted or substituted, alkyl, cycloalkyl, alkenyl, alkynyl, or aromatic esters of unsubstituted or substituted gentisyl alcohol or gentisyl quinone.

The present invention further relates to a method of producing a compound of formula I. The method involves reacting a compound of formula II 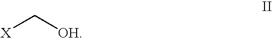
where X represents 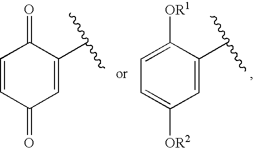
where R¹ and R² are each H or alkyl, under conditions effective to produce a compound of formula I.

The compound of formula II may, for example, undergo an acylation reaction, to produce a compound of formula I. The acylation reaction may involve, for example, an acyl halide, such as, for example, isovaleryl chloride.

Compounds of formula I where X represents 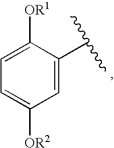
may further undergo oxidation to produce compounds of formula I where X represents

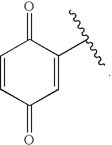

Compounds of formula I are useful in the management of cockroaches, and in particular, the German cockroach, Blattella germanica. Compounds of formula I are particularly useful as an attractant for cockroaches, and in particular, the German cockroach, Blattella germanica. Compounds of formula I may be used, for example, in any known method or apparatus of trapping, monitoring, or controlling populations of cockroaches. Indeed, the similarity between compounds of formula I to the recently isolated sex pheromone of the German cockroach B. germanica, indicates the potential usefulness of compounds of formula I in the management and control of cockroach populations. Nojima et al., “Identification of the Sex Pheromone of the German Cockroach, Blattela germanica”, Science 307:1104-1106 (2005), which is hereby incorporated by reference in its entirety.

In another aspect, the present invention relates to a method of attracting cockroaches, which involves exposing cockroaches to a trap charged with an effective attractant amount of a compound of formula I.

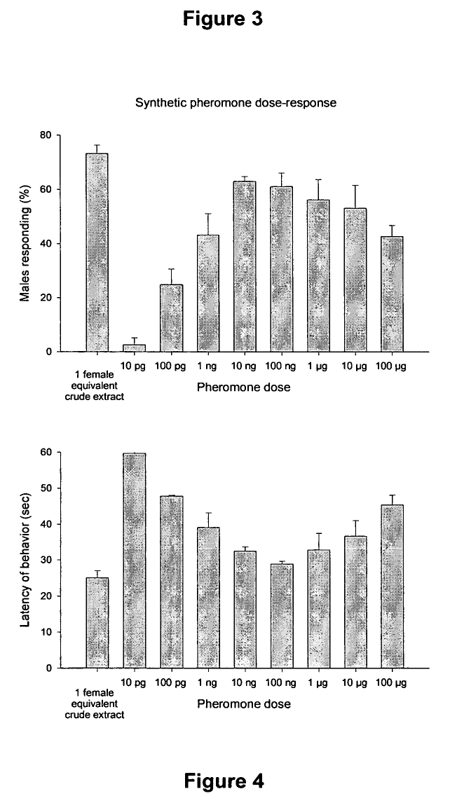

Generally, the method involves exposing the insects to a trap charged with a suitable amount of a compound of the present invention, for example, from about 1×10⁻⁴ to about 1×10⁴ μg, depending on the substrate used, preferably about 1×10⁻³ to about 1×10³ μg. In these amounts, the attractant lures the cockroaches to the trap, where they are restrained and/or killed, thus removing the trapped cockroach from the general population. Lower amounts of a compound of the present invention may be required on less retentive substrates, such as filter paper (as described in Example 4, and shown in FIGS. 3 and 4).

Cockroach traps according to the present invention may take any known form, and, in addition to the compound of the present invention, typically include a support and a cockroach restraint. Useful methods of restraint may include, for example, physical restraint or entrapment of the cockroach.

The support can be formed from any material suitable for supporting the attractant and the restraint. The support can be flexible, such as made from a cotton or canvas-like material, or rigid. Often, the support is in the form of a rigid, inexpensive material such as cardboard. The cockroach restraint may, for example, be an adhesive material, such as, for example, glue or tanglefoot, which is placed on the support in conjunction with the attractant.

The cockroach restraint may, for example, be a mechanical barrier, such as a door or flap constructed to allow cockroaches to enter the trap but not exit. Another type of mechanical restraint is known generally as a “pit-fall” trap, where the insects are attracted to and fall through an opening into, for example, water or some other container that prevents escape.

Further details regarding the use and construction of cockroach traps using adhesives and/or mechanical barriers are disclosed in, for example, Schal et al., “Integrated Suppression of Synanthropic Cockroaches,” Annu. Rev. Entomol. 35:521-551 (1990); Ballard et al., “The Effect of Selected Baits on the Efficacy of a Sticky Trap in the Evaluation of German Cockroach Populations,” J. Kans. Entomol. Soc. 55:86-90 (1982); Barak et al., “Using Attractant Traps to Help Detect and Control Cockroaches,” Pest Control 14:14-20 (October 1977); Bell et al., “Attraction of American Cockroaches (Orthoptera: Blattidae) to Traps Containing Periplanone B and to Insecticide-Periplanone-B Mixtures,” Environ. Entomol. 13:448-450 (April 1984); Owens, “Detection and Monitoring,” in, Rust et al., eds., Understanding and Controlling the German Cockroach, Oxford University Press, New York, pp. 93-108 (1995); Nalyanya et al., “Evaluation of Attractants for Monitoring Populations of the German Cockroach (Dictyoptera: Blattellidae),” J. Econ. Entomol. 94: 208-214 (February 2001), which are hereby incorporated by reference in their entirety.

The compounds of the present invention can also be used in conjunction with an insecticide. Because insecticides are generally repellent to cockroaches, the combination of both an attractant, such as a compound of the present invention, and an insecticide can have enhanced effectiveness over use of an insecticide alone. Useful insecticides include, for example, compounds containing inorganic insecticides (such as, for example, boric acid, diatomaceous earth, or silica gel), organophosphates (such as, for example, diazinon, chlorpyrifos, propetamphos, or acephate), carbamates (such as, for example, propoxur), pyrethroids (such as, for example, cypermethrin or cyfluthrin), insect growth regulators (such as, for example, hydroprene or lufenuron), nicotinoids (such as, for example, imidacloprid), phenyl pyrazoles (such as, for example, fipronil), sulfonamides (such as, for example, sulfluramid), microbials (such as, for example, Bt or spinosyns), and botanicals (such as, for example, limonene). The insecticide can, for example, be in the form of a spray (such as, for example, emulsifiable concentrates, wettable powders, or the like), aerosol, dust, bait, granular formulation, laminated slow release formulation, or any other suitable form.

The compound of the present invention may be combined with the insecticide and used without a support. Likewise, the compound may be combined with an insecticide on a support without a restraint, such as, for example, as in a product such as the “Lure n' Kill™ Killing Station” (commercially available from Hercon Environmental Co., Emigsville, Pa., USA). The combination of an insecticide and an attractant compound of the present invention may also be used in any of the trap configurations described herein, for example, with a cockroach restraint.

Further details regarding the combination of attractants and insecticides in sprays or in cockroach traps are described, for example, in Rust et al., “Increasing Blatticidal Efficacy with Aggregation Pheromone,” J. Economic Entomol. 70:693-696 (December 1977) and Bell et al., “Attraction of American Cockroaches (Orthoptera: Blattidae) to Traps Containing Periplanone B and to Insecticide-Periplanone-B Mixtures,” Environ. Entomol. 13:448-450 (April 1984), which are hereby incorporated by reference in their entirety.

In addition to use in traditional traps, the compounds of the present invention may also be used as an attractant in conjunction with cockroach biocontrol agents. In the context of the present invention, “biocontrol agent” refers to any biological enemy, such as, for example, predators, pathogens, or parasites, of cockroaches. Examples of biocontrol agents useful in connection with compounds of the present invention include, for example, pathogenic nematodes, fungi, yeast, bacteria, and viruses. In use, the attractant lures cockroaches to a dissemination station (or to isolated biological agents, if sprayed), where the cockroaches are infected with the biocontrol agent. A dissemination station is a location to which insects are attracted and infected with a biocontrol agent. The cockroaches then return to group aggregations and disseminate the agent to the rest of the population. As a result, an entire infestation of cockroaches can be reduced or eliminated by luring and infecting but a few members of the population with the appropriate pathogen.

The compounds of the present invention may also be used in concert with repellents to draw cockroaches away from sensitive structures, such as, for example, hospitals or restaurants. Steltenkamp et al., “Aryl and Alkyl Neoalkanamides: Highly Effective Insect Repellents,” J. Med. Entomol. 29:141-149 (March 1992); Nalyanya et al., “Integration of Repellents, Attractants, and Insecticides in a “Push-Pull” Strategy for Managing German Cockroach (Dictyoptera: Blattellidae) Populations,” J. Med. Entomol. 37:427-434 (May 2000), which are hereby incorporated by reference in their entirety.

In yet another aspect, the present invention relates to a method of disrupting cockroach mating, which involves exposing a cockroach population to an effective attractant amount of a compound of formula I.

This may be done, for example, by exposing a cockroach population to a compound of the present invention, in a quantity sufficient to cover the pheromone emissions by associated female cockroaches, and thereby prevent potential mates from finding each other, thus disrupting the ability of the cockroaches to mate.

The present invention is further described by reference to the following examples, which are provided by way of illustration, and not limitation.

### EXAMPLES

### Example 1

### Preparation of Insects

Blattella germanica was maintained at 27±1° C. under a 12 h light/12 h dark phtoperiod with rat chow (Purina #5012) and water provided ad libitum. Newly emerged adult males and females were collected daily and maintained in separate groups under the same conditions.

### Example 2

### Natural Pheromone Extraction

The last abdominal tergite of 3-7 day old virgin adult females was removed under the microscope and placed in methylene chloride. Thousands of such extracts were accumulated.

Crude pheromone extract was chromatographed on a mini SiO₂ column, by eluting successively with mixtures of pentane-ether in a sequence of 100% pentane, and 5%, 10%, 20% and 40% ether in pentane and 100% ether. The 40% ether in pentane fraction showed significant behavioral and electrophysiological activities, and this fraction was subjected to preparative high performance liquid chromatography (“HPLC”).

The active fraction was carefully concentrated under N₂ stream in an ice bath, then the inside wall of the vial was rinsed with a small amount of hexane. All of the concentrated fraction was subjected to SiO₂ HPLC purification. The system comprised a Rainin Rabbit-HP HPLC system (Rainin Instrument Co., Inc., MA, USA) equipped with a Rheodyne injector with 1.0 ml sample loop (Rheodyne, Inc., CA, USA). The column was an Econosil Silica 5 mm, 250×4.6 mm column (Alltech Associates, Inc., Deerfield, Ill., USA). The solvent system comprised a hexane-ether gradient, program 5% ether in hexane for 5 min, then increased linearly to 30% at 0.5%/min, to 100% at 5%/min, then it was kept for 20 min. A Dynamax UV-1 (Rainin Instrument Co., Inc., MA, USA) detector was used to monitor the eluate at 210 nm.

Effluent was collected every 3 min between 0 and 24.0 min and from 46.0 to 73.0 min, and every 1 min from 24.0 to 46.0 min. The active fraction was subjected to coupled gas chromatography-electroantennographic detection (“GC-EAD”) analysis and gas chromatography-mass spectrometry (“GC-MS”) analysis and further purification using preparative gas chromatography (“GC”).

For preparative GC, an HP 5890 gas chromatograph modified to a preparative GC equipped with a non-polar Equity-1 mega-bore capillary column (1.5 μm film thickness, 0.53 mm ID×5 m, Supelco Inc., Bellefonte, Pa., USA) was used. Nitrogen was used as the carrier gas at a head pressure of 6.9 kPa and a flow rate of 8.0 ml/min. The time for splitless injection was 1.0 min. The oven temperature was set initially at 40° C. for 2 min, increased at 10° C./min to 250° C., and held for 10 min. The injector and collection port temperatures were set at 150° C., and the septum purge flow rate was set at 1.5 ml/min with a total flow rate of 100 ml/min.

Active HPLC fraction was injected and the pheromone eluted from the outlet of the preparative GC was collected. The isolated pheromone component was subjected to nuclear magnetic resonance (“NMR”) analysis.

For GC-EAD analysis, a Hewlett Packard 5890 series II gas chromatograph equipped with a non-polar Equity-1 capillary column (0.25 μm film thickness, 0.25 mm ID×30 m, Supelco, Inc., Bellefonte, Pa., USA) or a slightly polar EC-20 capillary column (0.25 μm film thickness, 0.25 mm ID×30 m, Alltech Associates, Inc., Deerfield, Ill., USA) was used for analysis in splitless mode. Nitrogen was used as the carrier gas at a head pressure of 79 kPa (flow rate, 1.0 m/min). The oven temperature was programmed 40° C. for 2 min, increased at 15° C./min to 250° C. and held for 10 min. Injector and detector temperatures were set at 150 and 250° C., respectively.

The column effluent was combined with nitrogen make-up gas (30 ml/min) and then split 1:1 to the flame ionization detector (“FID”) and electroantennographic detection (“EAD”). The EAD outlet was secured in a charcoal-filtered and humidified air stream, refrigerated by a modified condenser flushed with 0° C. water, flowing at 500 ml/min over the antennal preparation.

A live male was fixed on a custom-made acrylic holder and used for EAD recordings. The tips of antennae were brought into contact with a capillary tube on the holder, which was filled with saline. A pure gold recording electrode was connected to the capillary tube, while the indifferent electrode was stuck directly into the mouth of the male. The holder was placed inside of the cooling condenser and maintained at about 5° C.

The output signal from the antennae was amplified by customized high-input impedance DC amplifier and was filtered by a high-pass filter with a cutoff frequency of about 0.5 Hz. The signals were recorded on an HP 3390A integrator synchronized with the GC integrator. GC-EAD analyses of the active fractions consistently revealed a single EAD-active compound.

The active fractions were subjected to GC-MS analyses on a Shimadzu GC-17A equipped with 15 m×0.25 mm ID; 0.25 mm DB-Sms (J&W Scientific, Inc., Folsom, Calif., USA) in splitless mode and coupled to a Shimadzu QP-5050 quadrupole mass spectrometer running in the electron ionization (“EI”) (electron ionization at 70 eV) scan mode. Helium was used as the carrier gas at a head pressure of 4.9 kpa (flow rate, 1.0 ml/min). Oven temperature was programmed 40° C. for 2 min, increased at 5° C./min to 220° C. and held for 5 min, and injector and interface temperature were set at 150° C. and 220° C., respectively.

GC-EIMS: the EAD-active compound showed a base peak at m/z 57 (100%) and characteristic ions at m/z 60 (68%), 122 (24%), 138 (16%), 150 (1.7%), 152 (1.3%), 162 (2.7%), 176 (2.0%), 180 (7.1%), 222 (0.9%) and 224 (1.1%). GC-CIMS: the EAD-active compound showed a set of characteristic ions at m/z 223 (51%), 251 (27%) and 263 (8.9%).

### Example 3

### Synthesis of Synthetic Pheromone

Both the EI and CI mass spectra observed for the natural pheromone indicate a molecular weight of 222 g/mol (m/z 222, EI and m/z 223, CI), thus suggesting a number of likely molecular formulas, which include C₁₂H₁₄O₄, C₁₃H₁₈O₃, C₁₄H₂₂O₂, C₁₅H₂₆O, C₁₅H₁₀O₂, C₁₆H₁₄O, and C₁₆H₃₀, and C₁₇H₁₈, among others. Fragmentation in the EI mass spectrum gives little useful information to pare down the number of formulas.

The 600 MHz ¹H NMR spectrum shows, with a rather high degree of certainty, that the compound has 14 hydrogens suggesting either C₁₂H₁₄O₄, or C₁₆H₁₄O. The first formula has 6 degrees of unsaturation while the latter has 10 degrees of unsaturation. Since it is difficult to draw “reasonable” structures with the second formula, C₁₂H₁₄O₄ was selected as the most likely formula for the pheromone.

The presence of an isopropyl group is evident in the 600 MHz NMR spectrum. There is a six proton doublet at δ 0.786 and a one proton multiplet centered at δ 1.987. This methine multiplet is obviously not a first order multiplet; it is clearly coupled to one or more protons in addition to the six methyl protons. A likely candidate is the two proton “doublet” at δ 1.920, which is distorted and “leaning” in the direction of the methine group. Assuming that the methine and methylene groups are coupled, a rough calculation of Δν/J gives an approximate value of 5. This calculation indicates that the patterns should not be first order but that they should retain some resemblance to first order.

The chemical shifts and coupling patterns are reminiscent of esters of isovaleric acid and, in fact, matches NMR spectra of known isovalerate esters. This piece of the structure uses C₅H₉O₂ and accounts for one of the degrees of unsaturation. The other piece, the alcohol moiety, must have a formula of C₇H₅O₂, noting that the oxygen atom associated with the alcohol portion has already been accounted for in the acid portion.

Thus, a good place to start on the alcohol piece in the NMR spectrum is the two proton doublet at δ 4.721. The coupling constant is small (˜2 Hz), which is too small for typical vicinal coupling in a freely rotating system, meaning either long range coupling or restricted rotation. Since the remaining piece is highly unsaturated, the likely case is that the small coupling is due to long range coupling. The chemical shift of this methylene group indicates that it is deshielded by an oxygen atom and by some other group, likely some form of unsaturation.

The remaining three protons in the NMR spectrum are likely either olefinic or shielded aromatic protons. The proton at δ 6.356 is an apparent quartet, which is explained by long range coupling to the methylene group at δ 4.721 and a similar (in magnitude) long range coupling to one of the other olefinic protons. The other two protons form a multiplet at δ 5.928. This multiplet can be dissected into (1) vicinal coupling to give an AB multiplet, almost a quartet (Δν/J˜1.7) and (2) long range coupling of one of the AB protons to the proton at δ 6.356.

This NMR pattern is consistent with a substituted para-benzoquinone. The primary alcohol of p-benzoquinone is gentisyl alcohol, and the corresponding quinone is gentisyl quinone:

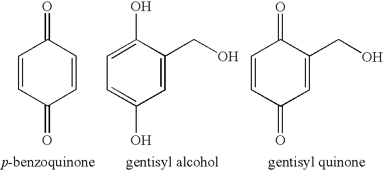

Thus gentisyl quinone isovalerate was proposed as the structure for the pheromone compound.

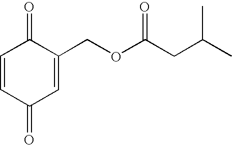

Since this is a new compound, and since there are no recorded chemical shifts of gentisyl esters in deuterobenzene, proof of structure is dependent on chemical synthesis. An unambiguous synthesis is outlined in Scheme I and its accompanying text.

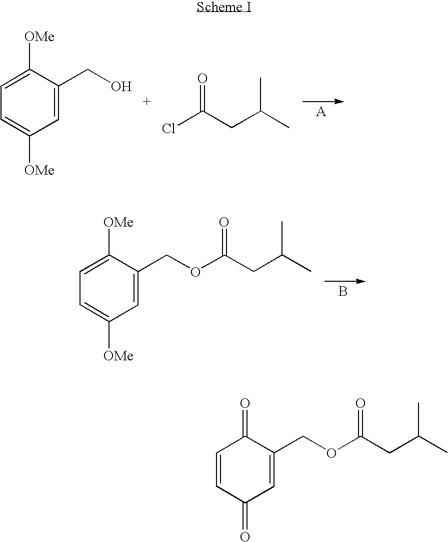

A. Acylation. As shown in Scheme 1, isovaleryl chloride (6.03 g, 50 mmole) was added to a solution of 2,5-dimethoxybenzyl alcohol (5.05 g, 30 mmole), pyridine (3.95 g, 50 mmole), and DMAP (5 mg) in 20 ml of CH₂Cl₂ at 0° C. with magnetic stirring. After the addition was complete, the mixture was stirred for another 2 h; saturated sodium bicarbonate was added to remove excess acid chloride. The mixture was extracted with ether three times, and the extract was washed with brine and dried over anhydrous sodium sulfate. The solvent was removed by rotary evaporator, leaving 7.4 g of crude ester.

B. Oxidation. Crude ester from A (2.0 g, 8 mmole) in 40 ml of acetonitrile was added to a solution of Ce(NH₄)₂(NO₃)₆ in 50 ml of water at room temperature with magnetic stirring. After the addition, the mixture was stirred for an additional 0.5 h and extracted with CH₂Cl₂. Methylene chloride was removed by evaporator, and the residue was redissolved in ether. The solution was stirred with aqueous sodium bicarbonate, the ether extract was washed with brine and dried over anhydrous sodium sulfate.

The crude quinone ester (1.3 g) was purified by flash chromatography; silica gel was eluted with ethyl acetate/hexane (1:8) to give 0.7 g of purified quinone ester. The compound was recrystallized from hexane-ether (2:1).

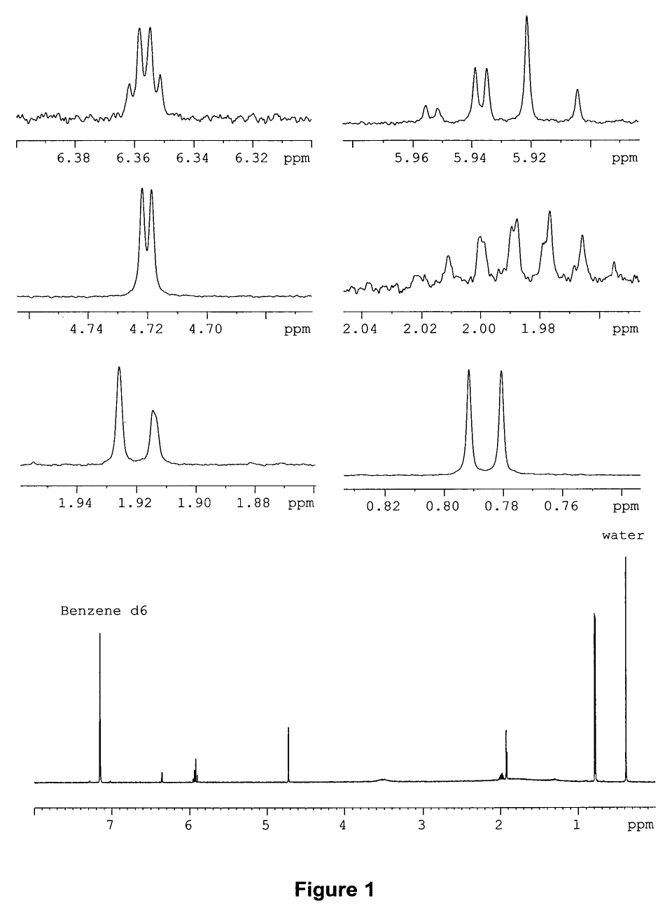

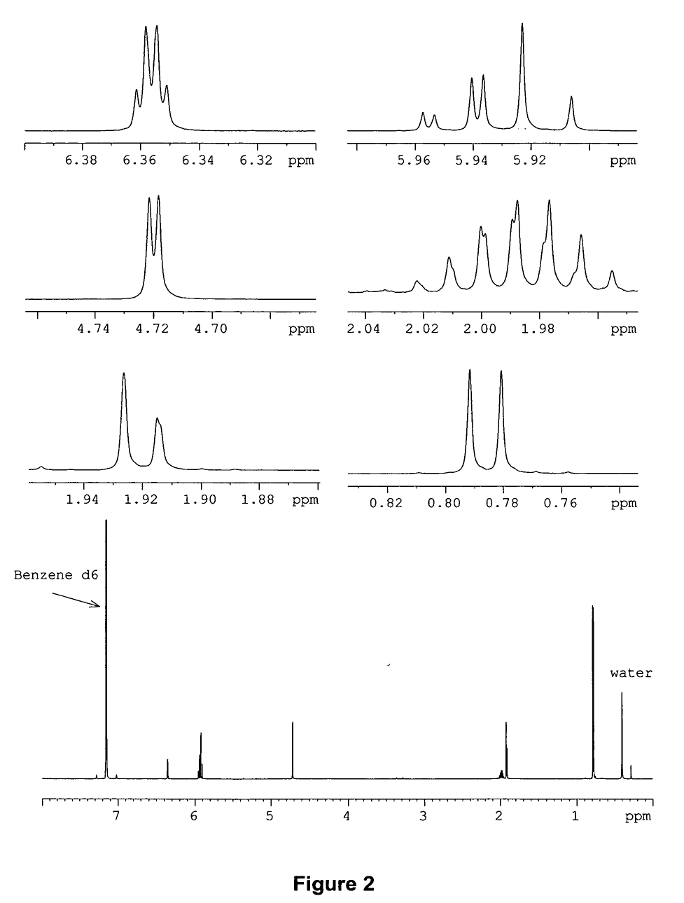

Comparative data for the natural product and the synthetic compound are shown in FIGS. 1 and 2, respectively.

### Example 4

### Behavioral Assays and Electroantennograms

The biological activity of extracts and fractions was assayed both behaviorally and with an electroantennogram (“EAG”). Schal et al., “Site of Pheromone Production in Female Supella longipalpa (F.) (Dictyoptera: Blattellidae): Behavioral, Electrophysiological, and Morphological Evidence,” Annals Entomol. Soc. Amer. 85:605-611 (1992), which is hereby incorporated by reference in its entirety. For behavioral assays a two-choice olfactometer was used. Liang et al., “Effects of Pheromone Concentration and Photoperiod on the Behavioral Response Sequence to Sex Pheromone in the Male Brown-Banded Cockroach, Supella longipalpa,” J. Insect Behavior 3:211-223 (1990), and Liang et al., “Volatile Sex Pheromone in the Female German Cockroach,” Experientia 49:324-328 (1993a), which are hereby incorporated by reference in their entirety. Briefly, a straight Plexiglas tube (55 cm long and 3 cm ID) was divided along 15 cm of its upwind end and air was drawn through it at 20 cm/min. A single cockroach was placed in a screen-gated cage at the downwind end 30 min before the start of an assay. A candidate attractant and control were introduced simultaneously at the upwind end of the olfactometer. The percentage of insects running upwind and the latency, in seconds, before they responded were recorded.

EAG activity was recorded as described in Example 2.

As seen in FIG. 3, males exhibited a clear dose-response to the synthetic pheromone. More than 60% of the males responded to 10-100 ng of the pheromone on filter paper. FIG. 4 shows that the latency of the male response reflected the dose-response pattern. Males exhibited the shortest latency (in sec.) between 10 ng and 1 μg.

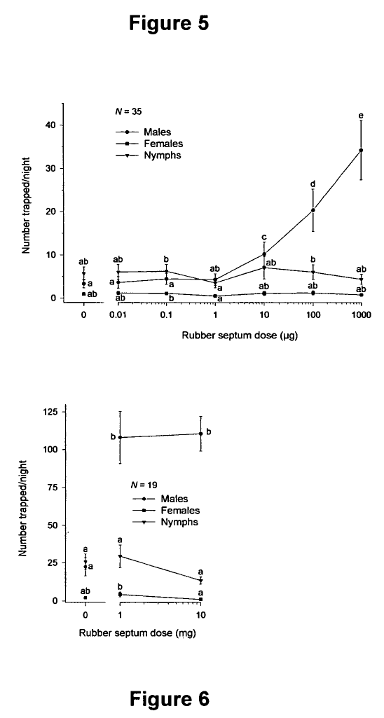

Field tests were also performed. Extracts of candidate compounds were loaded into rubber septa and placed in 1 pint mason jars, the inner walls of which were covered with petroleum jelly. Field tests were conducted along walls of an infested swine farm. To study the field dose-response, a completely randomized Latin-square design was used with 6 doses, control, and 5 replicates per dose. The trial was conducted in May-June, 2003. Overnight trap catch was enumerated by sex and stage. Data were log-transformed and analyzed by multi-way ANOVA (SAS). A separate dose-response study was repeated at higher doses to extend the dose response. Results are shown in FIGS. 5 and 6.

The compound of the present invention, a synthetic pheromone, represents an innovative approach to detection, monitoring and pest control, while minimizing risks of pesticide use to humans and the environment.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A compound of formula I: 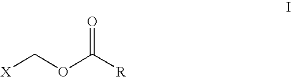
wherein:
R is selected from the group consisting of unsubstituted or substituted, alkyl, cycloalkyl, alkenyl, alkynyl, and aryl; and
X is 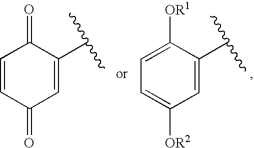
wherein R¹ and R² are each H or alkyl.

2. The compound according to claim 1, wherein R is alkyl.

3. The compound according to claim 2, wherein R is isobutyl.

4. The compound according to claim 1, wherein X is

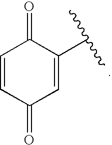

5. The compound according to claim 4, wherein R is isobutyl.

6. A method of producing a compound of formula I 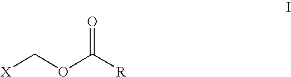
wherein:
R is selected from the group consisting of unsubstituted or substituted, alkyl, cycloalkyl, alkenyl, alkynyl, and aryl; and
X is 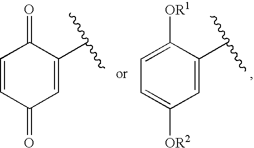
wherein R¹ and R² are each H or alkyl, said method comprising:
reacting a compound of formula II 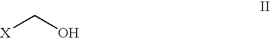
wherein:
X is 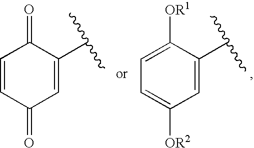
wherein R¹ and R² are each H or alkyl, under conditions effective to produce a compound of formula I.

7. The method according to claim 6, wherein the conditions effective to produce a compound of formula I comprise acylation of said compound of formula II.

8. The method according to claim 6, wherein X is 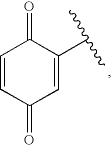
and said compound of formula I produced is

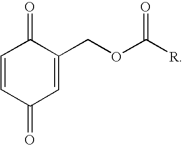

9. The method according to claim 8, wherein R is isobutyl, and said compound of formula I produced is

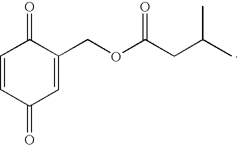

10. The method according to claim 6, wherein X is 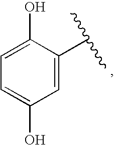
and said compound of formula I produced is

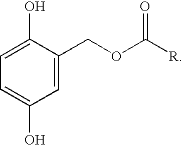

11. The method according to claim 10, wherein R is isobutyl, and said compound of formula I produced is

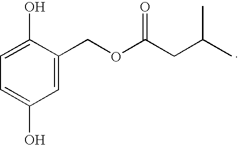

12. The method according to claim 11 further comprising:
oxidizing said compound of formula I to produce

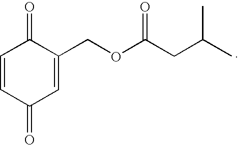

13. The method according to claim 6, wherein X is 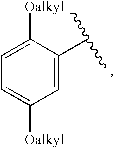
and said compound of formula I produced is

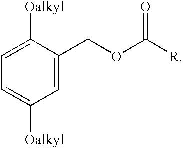

14. The method according to claim 13, wherein R is isobutyl, and said compound of formula I produced is

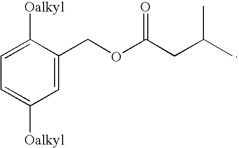

15. The method according to claim 14 further comprising:
oxidizing said compound of formula I to produce

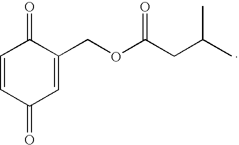

16. A compound which is an unsubstituted or substituted, alkyl, cycloalkyl, alkenyl, alkynyl, or aromatic ester of unsubstituted or substituted, gentisyl alcohol or gentisyl quinone.

17. The compound according to claim 16, which is

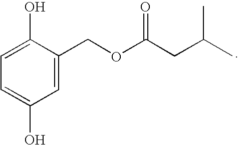

18. The compound according to claim 16, which is

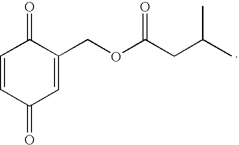

19. A method of attracting cockroaches comprising:
exposing said cockroaches to a trap charged with an effective attractant amount of a compound of formula I 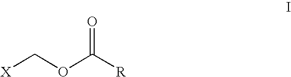
wherein:
R is selected from the group consisting of unsubstituted or substituted, alkyl, cycloalkyl, alkenyl, alkynyl, and aryl; and
X is 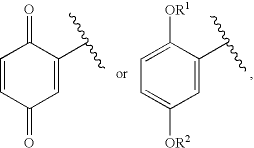
wherein R¹ and R² are each H or alkyl.

20. The method according to claim 19, wherein said compound of formula I is

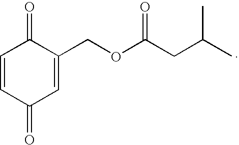

21. The method according to claim 19, wherein said cockroaches are of the type Blattella germanica.

22. A method of disrupting cockroach mating comprising:
exposing a cockroach population to a compound of formula I 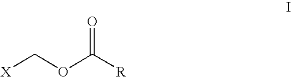
wherein:
R is selected from the group consisting of unsubstituted or substituted, alkyl, cycloalkyl, alkenyl, alkynyl, and aryl; and
X is 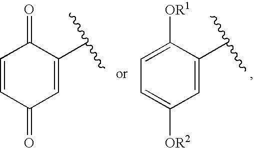
wherein R¹ and R² are each H or alkyl, in an amount sufficient to disrupt mating.

23. The method according to claim 22, wherein said compound of formula I is

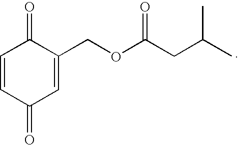

24. The method according to claim 22, wherein said cockroaches are of the type Blattella germanica.

25. A cockroach trap comprising:
a support;
a means for restraining cockroaches; and
a cockroach attractant amount of a compound of formula I 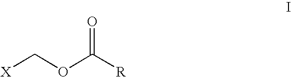
wherein:
R is selected from the group consisting of unsubstituted or substituted, alkyl, cycloalkyl, alkenyl, alkynyl, and aryl; and
X is 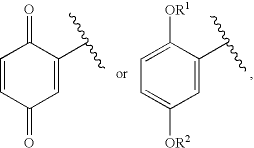
wherein R¹ and R² are each H or alkyl.

26. The cockroach trap according to claim 25, wherein said compound of formula I is

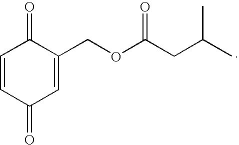

27. The cockroach trap according to claim 25, wherein said means for restraining cockroaches is mechanical.

28. The cockroach trap according to claim 25, where said means for restraining cockroaches is adhesive.

29. The cockroach trap according to claim 25 further comprising:
an insecticide.

30. The cockroach trap according to claim 25, wherein said cockroaches are of the type Blattella germanica.

31. A cockroach control system comprising:
an insecticide; and
a cockroach attractant having the formula I 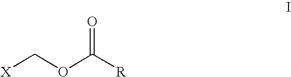
wherein:
R is selected from the group consisting of unsubstituted or substituted, alkyl, cycloalkyl, alkenyl, alkynyl, and aryl; and
X is 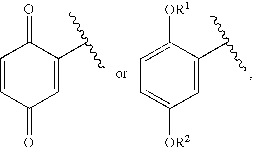
wherein R¹ and R² are each H or alkyl.

32. The cockroach control system according to claim 31, wherein said insecticide is selected from the group consisting of inorganic insecticides, organophosphates, carbamates, pyrethroids, insect growth regulators, nicotinoids, phenyl pyrazoles, sulfonamides, microbials, and botanicals.

33. The cockroach control system according to claim 32, wherein said insecticide is selected from the group consisting of boric acid, diatomaceous earth, silica gel, diazinon, chlorpyrifos, propetamphos, acephate, propoxur, cypermethrin, cyfluthrin, hydroprene, lufenuron, imidacloprid, fipronil, sulfluramid, Bt, spinosyns, and limonene.

34. The cockroach control system according to claim 31, wherein said system is placed on a support.

35. The cockroach control system according to claim 31, wherein said compound of formula I is

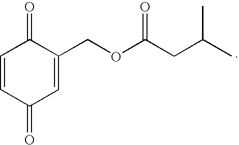

36. The cockroach control system according to claim 31, wherein said cockroaches are of the type Blattella germanica.

37. A cockroach control system comprising:
a biocontrol agent; and
a cockroach attractant having the formula I 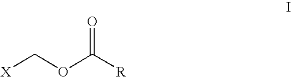
wherein:
R is selected from the group consisting of unsubstituted or substituted, alkyl, cycloalkyl, alkenyl, alkynyl, and aryl; and
X is 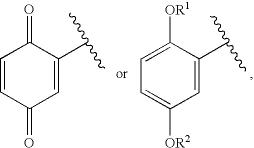
wherein R¹ and R² are each H or alkyl.

38. The cockroach control system according to claim 37, wherein said biocontrol agent is selected from the group consisting of pathogenic nematodes, fungi, yeast, bacteria, and viruses.

39. The cockroach control system according to claim 37, wherein said system is placed on a support.

40. The cockroach control system according to claim 37, wherein said compound of formula I is

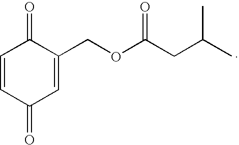

41. The cockroach control system according to claim 37, wherein said cockroaches are of the type Blattella germanica.
